(19) United States
(12) Patent Application Publication
(10) Pub. No.: US 2014/0034534 A1
(43) Pub. Date: Feb. 6, 2014

(54) SNAP-BACK BIASED DISCRETE CONSUMER PRODUCTS AND STERILIZATION WRAPS AND METHODS OF MANUFACTURING DISCRETE CONSUMER PRODUCTS

(71) Applicants: PERRY HANDZIAK, Belgium, WI (US); JON PELLAND, Sheboygan, WI (US); Henry L. Griesbach, III, Clarkston, GA (US); Keith J. Edgett, Middletown, DE (US)

(72) Inventors: PERRY HANDZIAK, Belgium, WI (US); JON PELLAND, Sheboygan, WI (US); Henry L. Griesbach, III, Clarkston, GA (US); Keith J. Edgett, Middletown, DE (US)

(73) Assignee: Curt G. Joa, Inc., Sheboygan, WI (US)

(21) Appl. No.: 13/565,199

(22) Filed: Aug. 2, 2012

(51) Int. Cl. A61B 19/02

(52) U.S. Cl. CPC A61B 19/026
USPC 206/438; 53/410; 206/525

(57) ABSTRACT

Discrete consumer products, such as sterilization wraps and methods for producing such discrete consumer products are disclosed. Sterilization wraps of the present disclosure avoid contacting exterior, non-sterile surfaces of sterilization wraps with the sterile contents of the sterilization wrap. Spring elements are coupled with the sterilization wrap to urge the sterilization wrap from a closed to an open position when desired. In this manner, the sterilization wrap avoids being re-introduced to the sterile equipment contained in the sterilization wrap.

### BACKGROUND OF THE INVENTION

This disclosure relates to methods of manufacturing snap-back biased discrete consumer products, and snap-back biased discrete consumer products produced by such methods.

Sterilization wraps are used to shield materials contained in the wraps, such as surgical tools, from contamination. The wraps are placed around the object(s) to be sterilized prior to sterilization. After sterilization, the contents may be used immediately or may be stored for use when desired.

A product that is made of several sheets of nonwoven plastic materials of various weights is used for wrapping trays of surgical instruments prior to sterilization. The wrapped tray of instruments may be placed in an autoclave unit and heated with steam to sterilize the package. The temperature in the autoclave is at or above the annealing temperature of the nonwoven plastic materials; therefore, the materials tend to form to the shape of the tray. That is, after the package is allowed to cool and is removed from the autoclave, the nonwoven materials have a tendency to remain in the shape that they were in when they were heated. This “memory” phenomenon hinders the package from properly opening and unfolding in its final stage of use.

If an exterior, non-sterile, surface of the sterilization wrap comes back into contact with the sterile contents, the sterile contents are no longer considered sterile. In a non-sterile condition, the contents cannot be used during surgical procedures. Therefore, it is desirable to avoid having non-sterile surfaces contact previously sterile surfaces and causing the use of an additional sterile tool.

### SUMMARY OF THE INVENTION

Snap-back biased discrete consumer products such as, but not limited to sterilization wraps used in surgical settings are described. Such sterilization wraps may be autoclavable. Other embodiments in which a snap-back biased discrete consumer product is desirable are also disclosed.

In one embodiment, the snap-back biased discrete consumer products are sterilization wraps used in surgical settings. Other embodiments in which a snap-back biased discrete consumer product is desirable are also disclosed. To aid in the unfolding process of a wrapped object, one or more spring elements can be added to the product. These spring elements can be positioned between or outside the nonwoven material sheets in specific locations such that when the product is in its final stage of use, the spring elements help overcome the resistance or “memory” of the folds in the nonwoven materials and bias the materials to unfold.

The disclosed embodiments are useful to avoid contacting exterior, non-sterile surfaces of sterilization wraps with the sterile contents of the sterilization wrap. Spring elements are coupled with the sterilization wrap to urge the sterilization wrap from a closed to an open position when desired, and which significantly reduces the laid-open bias to allow the sterilization wrap to remain in a concealing position. By being urged to remain in an open position, the sterilization wrap avoids being re-introduced to the sterile equipment contained in the sterilization wrap.

### BRIEF DESCRIPTION OF THE DRAWINGS

FIG. 1 is a top plan view of a sterilization wrap of the present disclosure, with spring elements positioned coupled to the sterilization wrap;

FIG. 2A is a side cross-sectional view of the sterilization wrap;

FIG. 2B is a side cross-sectional view of a portion of the sterilization wrap;

FIG. 2C is a side cross-sectional view of another portion of the sterilization wrap;

FIG. 3 is a perspective, in use view of the sterilization wrap in an open condition;

FIGS. 4-7 are perspective, in use views of the sterilization wrap during closing;

FIGS. 8, 9A, 9B and 10-11 are perspective, in use views of the sterilization wrap during opening;

FIG. 12 is a top plan view of an alternate embodiment of a sterilization wrap of the present disclosure, with an alternate spring element arrangement;

FIG. 13 is a top plan view of a second alternate embodiment of a sterilization wrap of the present disclosure, with a second alternate spring element arrangement.

FIG. 14 is a drawing of sterilization wrap having spring elements outside of the object landing area.

FIG. 15 is a drawing of sterilization wrap having spring elements outside of the object landing area as well as stiff regions dissociated from the object landing area and spring elements.

FIG. 16 illustrates a configuration of spring elements and stiff regions in conventional rectangular sterilization wrap.

FIG. 17 illustrates a configuration of stiff regions (without spring elements) in conventional sterilization wrap.

### DESCRIPTION OF THE PREFERRED EMBODIMENT

The disclosed embodiments are described in certain instances as relating to the snap-back biased discrete consumer products such as sterilization wraps used in surgical settings, but it is understood that other embodiments in which a snap-back biased discrete consumer product is desirable are also within the scope of the disclosure. Also, the manufacturing techniques described herein can be used on products other than sterilization wraps used in surgical settings, and in particular used to manufacture any consumer product for which a snap-back biased element is desirable.

Referring now to FIG. 1, a top plan view of a sterilization wrap 10 of the present disclosure is shown, showing with dashed lines where an object intended for concealment within the sterilization wrap 10 is outlined, the sterile object 12 generally defining an object landing area 11, with the remainder of the sterilization wrap generally defining an object covering area 13. Spring elements 14 are positioned coupled to material layer or layers 16 of the sterilization wrap 10. The spring elements 14 are desirably, but not necessarily of a flatter variety so that they are easily concealed either within or on one layer of material 16.

As shown in FIG. 1, the area where the spring elements 14 are attached (henceforth the “spring area”) may partially coincide with the object landing area 11, at least partially underlying the object 12. Alternatively, the spring area may not coincide with the object landing area 11 (FIG. 14) but would be wholly outside the object landing area and so would not underlay the object 12.

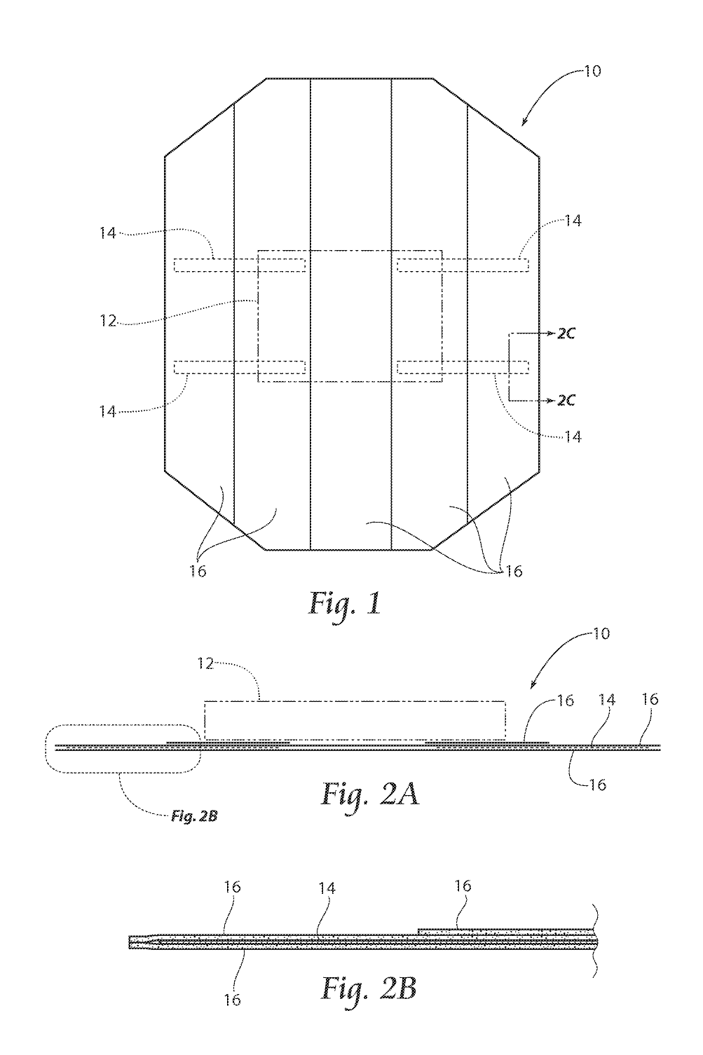

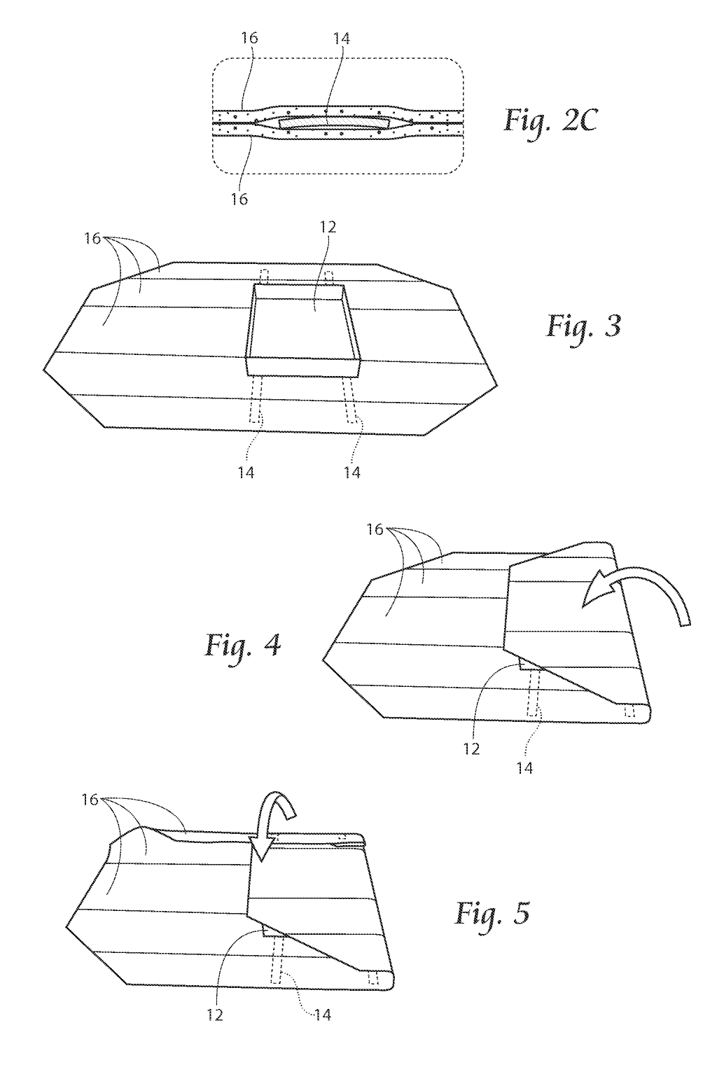

Referring now to FIG. 2A, a side cross-sectional view of the sterilization wrap 10 is shown, with the object landing area 11. In one embodiment, layers of material 16 can be provided in a configuration such that the layers of material 16 avoid building up a memory, which could cause the material 16 to act in an undesirable fashion (e.g. curling back to contact the object). Additionally, the layers of material 16 could be arranged to provide additional resistance to impact or penetration by providing additional layers 16 in areas expected to encounter more stress (e.g. corners, edges).

Referring now to FIG. 2B a close-up side cross-sectional view of a portion of the sterilization wrap 10 is shown. In this embodiment, the spring element 14 is provided concealed within material layers 16.

Spring elements 14 can be any material type, mounted between any of the material layers 16, or external to the material layers 16. Spring elements 14 can also be different lengths and configurations, either coincident with a material layer edge (not shown) or not coincident with a material layer edge (as shown in FIG. 2B). Also evident on FIG. 2B is that a plurality of material layers 16 can be provided as desired, to increase

Referring now to FIG. 2C, a side cross-sectional view of a spring element 14 is shown. In one embodiment, the spring element 14 comprises a somewhat flexible strip of metal or other flexible material, arranged in a cupped fashion. The spring elements can be made of strips of steel, aluminum or plastic material of similar size and shape of the steel used in self retracting tape measures. That is, the material can be formed in a curved shape across its width. These strips can be any length to meet the requirements for various product sizes and opening demands.

As shown in FIG. 2C, the spring element 14 may be a strip with a curved or cup-shaped cross-section in at least a portion of the strip where the direction of the cupshape is down, e.g. away from the object landing area (although a strip with the cup-shape up, or a different spring element arrangement could also be used). In this arrangement, the strip as the spring element 14 will bias or urge the material layers 16 to lay open (such as shown in FIG. 3). Once the strip 14 is past a “break open” position, or an over the top position, the spring element 14 or a portion thereof goes essentially limp, which significantly reduces the laid-open bias. This allows the sterilization wrap to remain in a concealing position (see FIG. 7).

The “break open” position is when the entirety of the spring element 14 is unstressed. For the strip embodiment, the break open position is when the spring element 14 maintains all its cup-shapes. The “over the top” position is when a portion of the spring element 14 overlays any part of the object landing area and any portion of the spring element 14 is stressed. For the strip embodiment for FIG. 2C, the over the top position is when at least one cup-shape configuration present in the strip at the break open position changes, e.g. becomes uncupped partially, completely, or inverted, while the strip at least partially overlays the object landing area. A perspective view of the sterilization wrap 10 in a laid open condition is shown in FIG. 3. In this condition, an object 12 can be placed in the object landing area 11 in the wrap 10 for concealment, or removed from the wrap 10 when ready for use in the sterile environment.

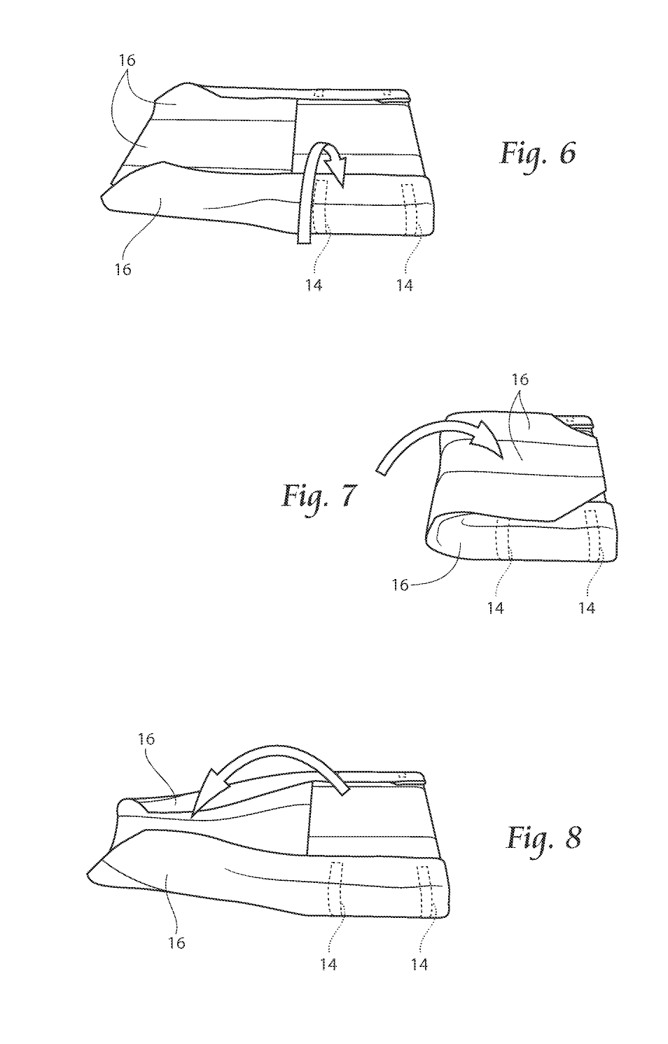

FIGS. 4-7 are perspective views that show the sterilization wrap 10 during closing from the open condition to the concealing position. Referring first to FIG. 4, in one method of use, a first edge of the sterilization wrap 10 is folded over the object 12 in the object landing area 11, with other edges of the wrap 10 folded over the object 12 sequentially, until the concealing position as shown in FIG. 7 is achieved. In this concealing position, the object 12 can be stored until ready for use.

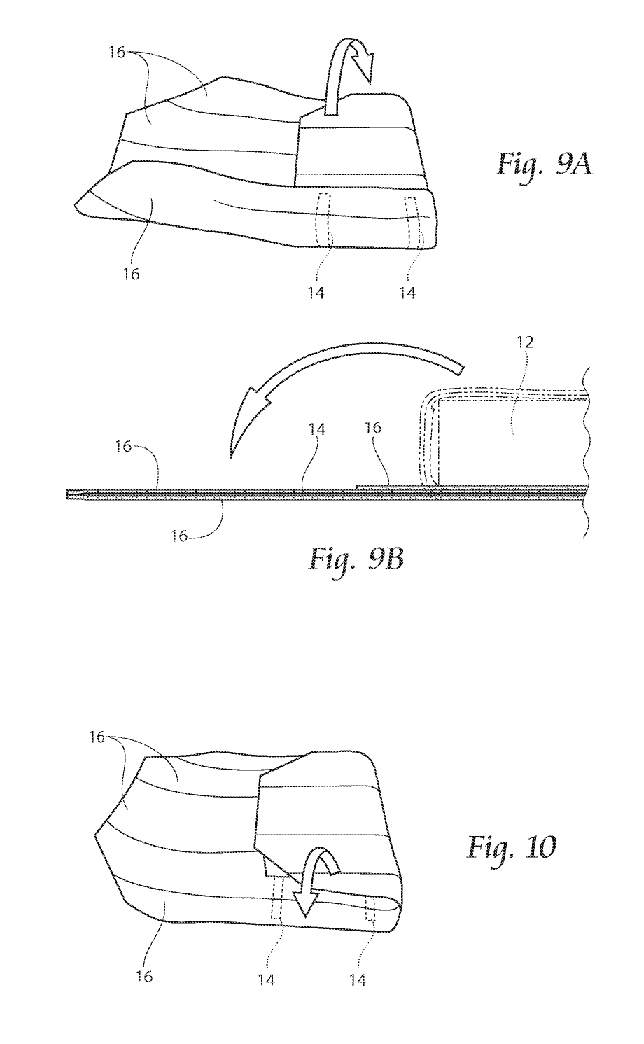

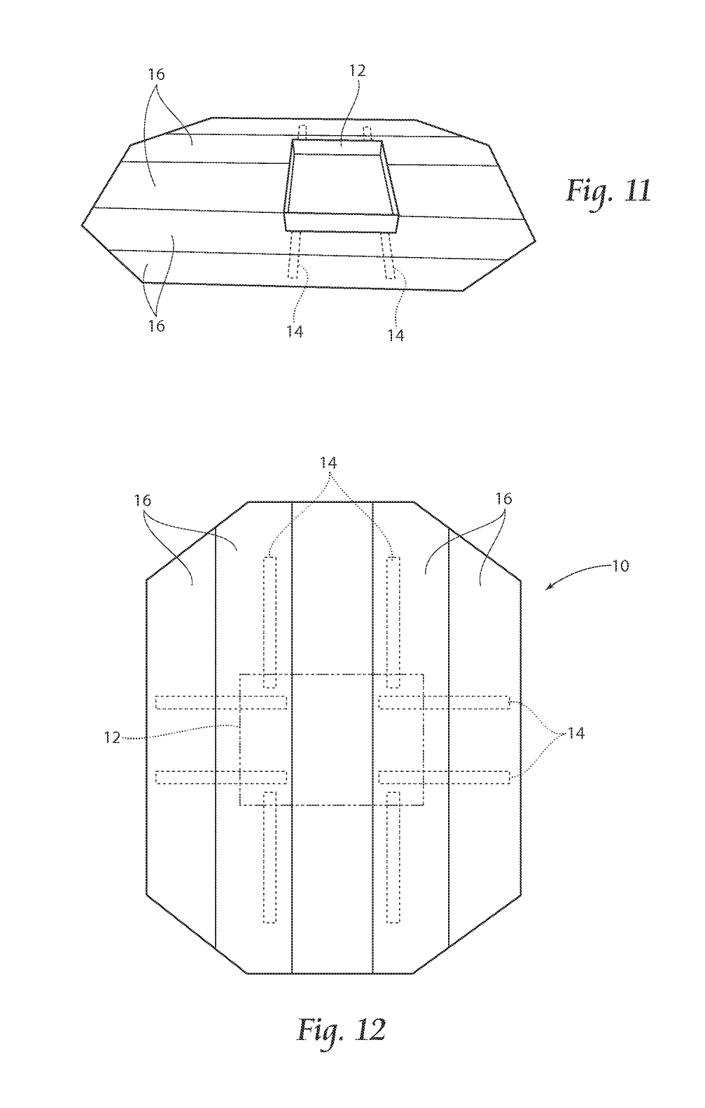

FIGS. 8, 9A, 9B, 10 and 11 are perspective and side views of the sterilization wrap 10 during its opening from the concealing position to the laid-open position. The unfolding of edges of the sterilization wrap 10 is accomplished in reverse order of the closing shown in FIGS. 4-7. Once the spring element or elements 14 revert back to the break open position, or past the break open position in a direction opposite the over the top position, the spring element 14 biases the sterilization wrap to the open position as shown in FIG. 11. By biasing the material layers 16 of the wrap 10 in the open position, the wrap 10 will, as a whole, usually lay flat in the open position as shown in FIG. 11 and contact of the object 12 by exterior, potentially non-sterile surfaces of the wrap 10 will be avoided.

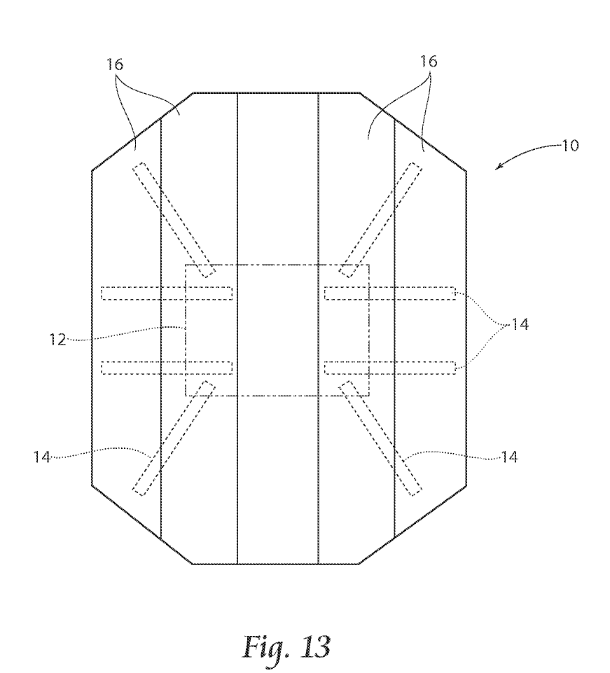

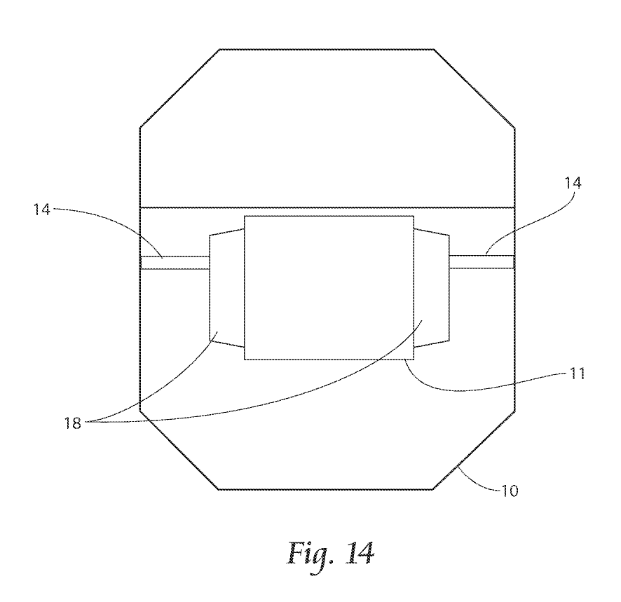

Referring now to FIGS. 12 and 13, top plan views of alternate embodiments of a sterilization wrap 10 of the present disclosure are shown. In FIGS. 12 and 13, alternate spring element 14 arrangements are provided, which allow for additional shapes of and directional biases.

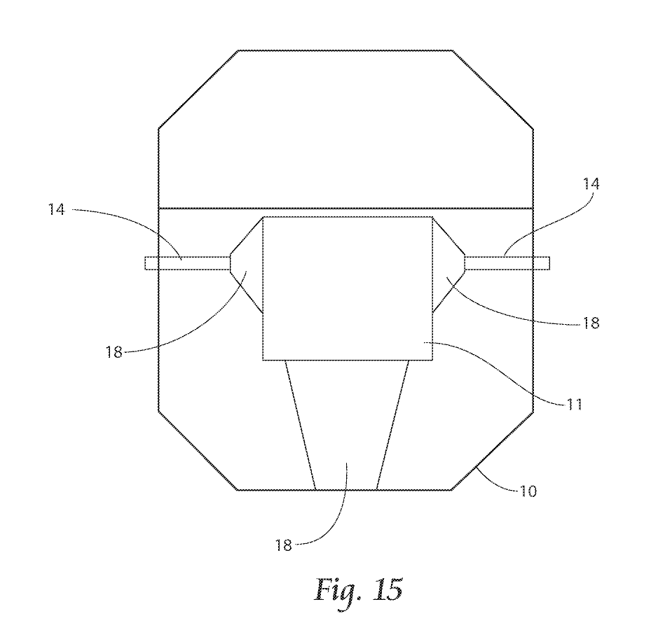

As shown in FIGS. 14 and 15, spring elements 14 may also be used outside of the intended object landing area 11 to actively overcome the resistance to unfolding of the sterilization wrap, as mentioned above. The spring elements 14, such as a thin strip of material that is flexible in the length dimension yet relatively inflexible in the width dimension, e.g. a thin metal or plastic strip that is slightly curved in the width direction, are positioned near, yet outside of, the object landing area 11 and attached to the sterilization wrap 10 so that a portion of the spring element 14 is bent over the object 12 when the wrap is folded around the object 12. The portion of the spring element 14 nearest the object landing area 11 does not initially interfere or retard folding of the wrap upwards but generally does in subsequent folding of the wrap.

Regions of sterilization wrap 10 that are relatively stiffer than other regions can be configured to further aid in unfolding. These stiff regions 18 can be of the same basis weight as neighboring regions or they can differ. The stiff regions 18 can serve to anchor the end portion of the spring element nearest the object landing area 11. The stiff regions 18 can also counteract the set that is imparted to the sterilization wrap 10 due to folding and sterilizing, thus promoting the wrap to return to a flat position after unfolding.

The stiff regions 18 may include continuous or discontinuous heat-fused or at least partially bonded areas of the sterilization wrap itself (e.g. geometric area shapes, lines), continuous or discontinuous heat set areas of sterilization wrap itself (e.g. thermal treatment to impart a biased curving opposition the direction of intended folding), added components attached to the sterilization wrap (e.g. flat layers, flexible elements with inherent arcing opposite the direction of intended folding), and/or their combinations.

FIGS. 14 and 15 also illustrate configurations of spring elements 14 and stiff regions 18 in a wrap assembly suitable for rectangular shaped objects 12 with minimum fold overlaps. The stiff regions 18 may function best with spring elements 14 for this type of wrap 10 when the stiff regions 18 do not extend beyond the sides of the object 12 to be wrapped while a portion of the spring element(s) does.

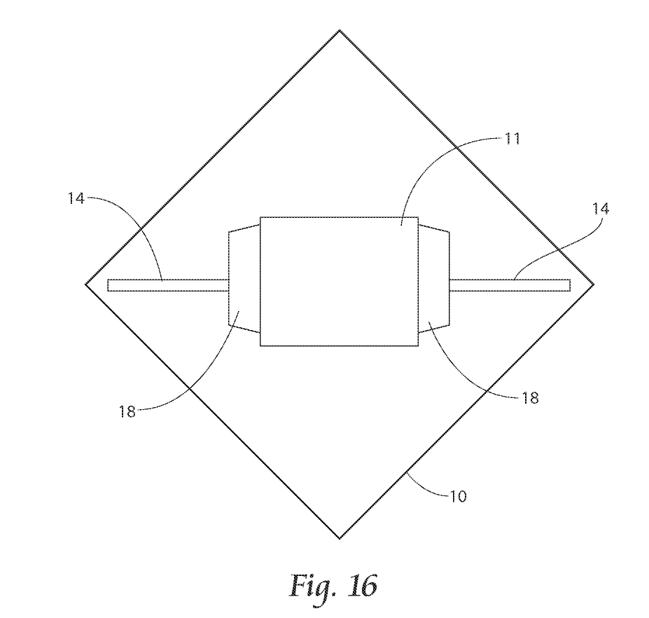

FIG. 16 illustrates the use of spring elements 14 with a conventional sterilization wrap. In this embodiment, the spring elements 14 may or may not underlay the object landing area 11. In addition, optional stiff regions 18 may also be used to help counteract the set that is imparted to the sterilization wrap 10 due to folding and sterilizing.

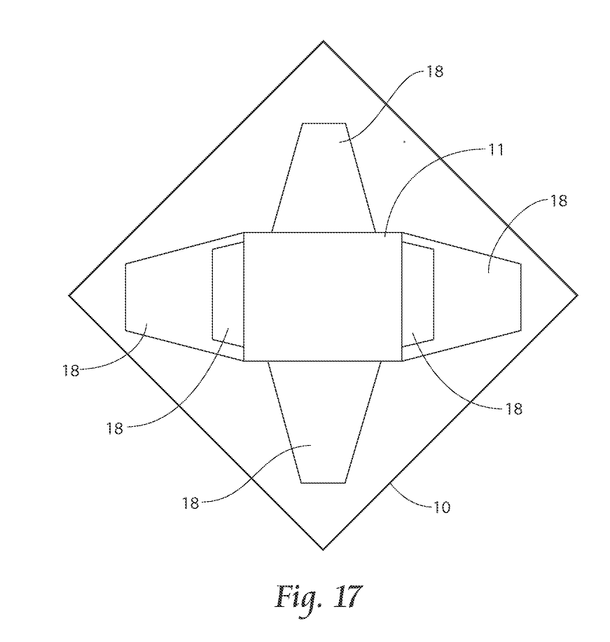

When stiff regions 18 are dissociated from spring elements 14, they can be involved in folds to counteract permanent creasing or at least diminish the set increases when they occur. This is shown in FIG. 17. Stiff regions 18 are shown with greater shading indicating a greater degree of stiffness.

To manufacture the wraps 10 of the present disclosure, a first material layer 16 is provided at a first speed. Spring elements 14 are severed and introduced to the material layer 16. Spring elements 14 are then coupled to the material layer 16. The coupling can be accomplished by provided an additional, overlying material layer 16 atop the spring elements 14, by providing or creating a seam in the material layer 16 into which a spring element 14 is provided, or by mechanically or adhesively coupling the spring element 14 to the material layer 14. Next, additional material layers 16 can be provided where desired.

Spring elements 14 can be introduced to a running web of material 16 by transverse introduction, or machine direction introduction of a rotated spring element 14. Angled introduction is also possible.

Introduction of spring element from a magazine (pre-cut) is possible, as is cut and place either in a transverse introduction where the material remains in that orientation, or rotated into place introduction (not shown). Methods such as those disclosed in U.S. Pat. No. 7,452,436 (incorporated herein by reference) can be used. Alternatively, a transverse application method can be used, as disclosed in U.S. Pat. No. 7,811,403, also incorporated herein by reference.

The material for the spring elements can be supplied in roll form and unwound and fed into the machine process using a conventional unwind system that can control the feed rate to provide a specified length of the material and to ensure that it is properly tensioned. After the material is unwound, adhesive is applied to one side and it is conveyed to a cut off unit where it is cut to the desired length. Once the spring element is cut to the proper length, it is picked up and held in place on a transfer drum. (The drum can use vacuum or other mechanical methods to hold the spring element material to the drum.) The spring element will be oriented such that the adhesive will be on the side of the spring element material that is facing away from the transfer drum.

The unwind equipment and transfer drum is placed in the machine line such that the spring element will be conveyed into the machine perpendicular to the direction of travel of the primary web to which the spring element will be attached. The spring elements will enter one side of the transfer drum, (the top, for example) and as the transfer drum rotates, the spring elements will be transported to the bottom, for example, position of the drum. When the first spring element is at the bottom of the transfer drum, the spring element will be positioned such that its adhesive coated side is in close proximity to the surface of the running primary web.

At a predetermined time, a dauber device rotates into a position that will cause the primary web to be deflected toward the spring element. As the dauber continues through its range of travel, the primary web will be deflected to an elevation at which it contacts the adhesive coated surface of the spring element with enough force that the adhesive securely bonds the spring element to the primary web. The primary web will pull the spring element away from the transfer drum and the spring element will continue to travel with the primary web. This sequence will be repeated for each subsequent spring element.

The process equipment used to apply the spring elements can be adjusted in the cross machine direction and multiple transfer drums can be positioned over the primary web.

In alternative embodiments, there are variations of the spring element material that can be accommodated with some reorientation of the process equipment and/or the addition of process units. These are listed below.

In a first alternative embodiment, the spring element material may be supplied in discrete pieces as opposed to roll form. This would require the roll unwinding system to be replaced with a feeding system.

In a second alternative embodiment, the spring element material may be flat as opposed to curved.

In a third alternative embodiment, the spring element material may be supplied flat and need to be formed into a curved shape on the machine. This would require that roll forming equipment be installed between the unwind system and the cut off knife. If the material is plastic, it may be necessary to use heated and chilled forming rolls.

In a fourth alternative embodiment, if the spring element material is curved, it may be applied with its concave side facing toward the primary web or it may be applied with its concave side facing away from the primary web.

In a fifth alternative embodiment, a cut off knife is used in conjunction with a die cutting unit to create radii (or other profiles) on the corners of the spring elements as they are cut to length.

In a sixth alternative embodiment, the spring element may be applied in the same direction as the running web by placing the transfer drum in line with the running web or by feeding the transfer drum at 90 degrees to the running web direction and rotating the cut spring element on the transfer drum to align it with the running web.

Alternative methods of adhesive application may also be employed. First, the adhesive may be applied to the entire surface of the spring element or it may be applied only in discrete areas on the spring element.

Second, the adhesive may be applied to the concave or the convex surface of a curved spring element.

Likewise, the orientation of the transfer drum may vary. In one embodiment, if the spring element needs to be applied to the underside of the primary web, the transfer drum would be placed below the primary web and the dauber would be placed above the web. The spring elements would be picked up by the drum at the bottom position and transported to the top position where they would be transferred to the primary web.

In a second embodiment, the surface of the transfer drum to which the spring element is held may be profiled to match the curvature of the spring element.

In a third embodiment, if the transfer drum is circular, the ends of the spring element will be further away from the primary web than the midpoint of the spring elements. This difference may cause the spring element to skew as it is being daubed onto the primary web. To compensate for this, the transfer drum diameter will be maximized. In addition, the transfer drum may be made in the shape of a polygon with sides that are similar in length to the spring element. This would result in the entire length of the spring element being at the same distance from the primary web when the dauber contacts the primary web.

In a fourth embodiment, it may be beneficial for the spring element to be attached to a carrier web before it is picked up by the transfer drum. The carrier web would then be attached to the primary web. The carrier web could facilitate transporting of the spring element and it could enable the same transfer drum diameter to be used to apply spring elements of different lengths without changes to the set up of the transfer drum. In addition, a carrier web could reinforce to the primary web so that the potential for the spring element to puncture the primary web is reduced. The carrier web may be single layer or it may be multiple layers with the spring element sandwiched between the layers. Alternatively, the spring element may be attached to the carrier web with adhesives, ultrasonic bonding, or a similar bonding technology; or the spring element may be captured in a pocket in a multilayer carrier web without the spring element being bonded to the carrier web.

The foregoing is considered as illustrative only. Furthermore, since numerous modifications and changes will readily occur to those skilled in the art, it is not desired to limit the disclosure to the exact construction and operation shown and described. Details may be changed without departing from the claimed invention.

I/We claim:

1. A method of making a flexible product with a snap-back feature comprising:
providing a first material layer comprising an object landing area for receiving an object to be covered, a first spring area, and an object covering area;
providing a first spring element coupled to said material layer at said spring area;
said spring element having both an unstressed condition for receiving said object to be covered, and a stressed condition defining a concealing position;
said object covering area foldable over said object to be covered;
at least a portion of said spring area overlying said object to be covered when said biased spring element is in said concealing position.

2. The method according to claim 1, said method further comprising providing a second object covering area foldable over said object to be covered.

3. The method according to claim 1, said method further comprising providing a second material layer sandwiching said spring element between said first and said second material layers.

4. The method according to claim 1, said method further comprising providing a third material layer overlying said object landing area and coupled to at least one of said first and said second material layers.

5. The method according to claim 1, said method further comprising providing a second spring area carrying a second spring element coupled to said material layer at said object covering area.

6. The method according to claim 1, said defining a lengthwise and a widthwise direction, said first spring element provided in a lengthwise direction.

7. The method according to claim 1, said product defining a lengthwise and a widthwise direction, said first spring element provided in a widthwise direction.

8. A product with a snap-back feature comprising:
a material layer comprising an object landing area for receiving an object to be covered, a spring area, and an object covering area;
a spring element coupled to said material layer at said spring area;
said spring element having both a laid-open for receiving said object to be covered, and a foldable concealing position; said object covering area foldable over said object to be covered;
at least a portion of said spring area overlying said object to be covered when said spring element is in said concealing position.

9. The product of claim 8 wherein said spring area at least partially coincides with said object landing area.

10. The product of claim 8 wherein said spring area is outside of said object landing area.

11. The product according to claim 8, said product further comprising a second object covering area foldable over said object to be covered.

12. The product according to claim 8, said product further comprising another material layer sandwiching said spring element between material layers.

13. The product according to claim 8, said product further comprising an additional material layer overlying said object landing area and coupled to at least one of said material layers.

14. The product according to claim 8, said product further comprising a second spring area carrying a second spring element coupled to said material layer at said object covering area.

15. The product according to claim 8, said product defining a lengthwise and a widthwise direction, said spring element provided in a lengthwise direction.

16. The product according to claim 8, said product defining a lengthwise and a widthwise direction, said spring element provided in a widthwise direction.

17. The product according to claim 8, said product defining a lengthwise and a widthwise direction, said spring element provided in a lengthwise direction and a widthwise direction.

18. The product according to claim 8 wherein said product is sterilization wrap.

19. The product according to claim 8 wherein said product is autoclavable.
